(19) United States
(12) Patent Application Publication
(10) Pub. No.: US 2009/0198289 A1
(43) Pub. Date: Aug. 6, 2009

(54) Fortified cannulated screw

(76) Inventor: Easton L. Manderson, Ashton, MD (US)

Correspondence Address: Curtis D. Kinghorn;Sirius Strategic, LLC, 6769 W. Shadow Lake Dr., Lino Lakes, MN, 55014, US

(21) Appl. No.: 12/012,377

(22) Filed: Feb. 2, 2008

(51) Int. Cl. A61B 17/56

(52) U.S. Cl. 606/304; 606/301

(57) ABSTRACT

A cannulated screw is provided having internal threads in a lumen to accommodate a fortifying screw. The fortifying screw is screwed into the threads in the lumen of the cannulated screw after the cannulated screw has been accurately placed in the bone by following a guide pin or guide wire to the desired location is as is commonly done with cannulated screws. The fortifying screw fortifies the cannulated screw and gives it strength approximately equal to that of a solid screw.

### BACKGROUND OF THE INVENTION

1. Field of the Invention

The present invention relates generally to methods and medical devices that connect together fractured bone or fuse bones of one or more joints.

2. Description of Related Art

Surgical screws are now commonly used to repair bone fractures or to fuse bones of a joint together. For example, in intertrochanteric fractures, that is, fractures of the femur below the neck of the femur, a common treatment is to use a screw and plate device to hold the two fragments in position while the fracture heals. In this process, the surgeon inserts a large screw into the femoral head across the fracture. The plate is positioned down the shaft of the femur and secured in place by smaller screws that pass into the femur.

Bone fractures of all types may occur where the fracture is located close to nerves or blood vessels. When placing screws into the bone to treat the fracture, it is important that the screws stay away from and do not damage the nerves or blood vessels. Therefore, accurate screw placement is important. One aid to surgeons in accurately placing screws has been the use of guide pins or guide wires.

Typically, under flouroscopy, a borehole is drilled into place in the bone. A guide pin or guide wire is inserted into the borehole where the guide pin or guide wire is held in place in the borehole by friction. A cannulated screw (i.e., a screw with a channel extending through the screw from one end to the other end) is placed over the guide pin or guide wire and inserted into the bone by screwing the screw into the bone. Once the screw is placed in the bone at the desired location, the guide pin or guide wire is removed. The use of cannulated screws over guide pins or guide wires has greatly simplified screw placement in repairing fractures.

But, small bones are generally not tolerant to multiple drill holes used to accurately and precisely place screws. As a result, it is desirable to make such drill holes as small as possible in order to preserve as much bone as possible. The requirement to use small drill holes means that the screws used in such holes must also be as small as possible.

In addition, many bones are weight-bearing. Where the screws used to heal fractures in such bone are small, weight-bearing may cause deformation of a traditional cannulated screw. For example, cannulated screws up to 7.0 mm in diameter lack the strength of solid screws of similar diameters because of their hollow cross section and long lengths Therefore, in some fractures where a cannulated screw could be placed accurately over a guide wire in order to aid in the placement of the screw, the lack of strength of the cannulated screw itself may make choosing such a cannulated screw unwise.

### SUMMARY OF THE INVENTION

A cannulated screw is provided having internal threads in a lumen to accommodate a fortifying screw. The fortifying screw is screwed into the threads in the lumen of the cannulated screw after the cannulated screw has been accurately placed in the bone by following a guide pin or guide wire to the desired location is as is commonly done with cannulated screws. The fortifying screw fortifies the cannulated screw and gives it strength approximately equal to that of a solid screw.

The disclosed cannulated screw system, as used in accordance with the methods of the invention, ensures a more effective function than prior art cannulated screws. It is, therefore a primary object of the present invention to provide an effective cannulated screw. Other objects of this invention, in one or more embodiments, are to:

minimize bone removal required to place the cannulated screw;

provide a cannulated screw that is easy to implant; and

provide a cannulated screw that can be used on a weight-bearing bone and bear the loads placed on such weight-bearing bone.

It is therefore an object of the present invention in one or more embodiments to provide a device that meets at least one of the objects listed above. Not all of these objects need be present in a single embodiment. Instead, a particular embodiment may have one or more of these objects. These and other objects of the invention will be clear from the following detailed description of the invention in connection with the drawings.

### BRIEF DESCRIPTION OF THE DRAWINGS

The invention will be described hereafter in detail with particular reference to the drawings. Throughout this description, like elements, in whatever embodiment described, refer to common elements wherever referred to and referenced by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All Figures are drawn for ease of explanation of the basic teachings of the present invention only; the extensions of the Figures with respect to number, position, relationship, and dimensions of the parts to form examples of the various embodiments will be explained or will be within the skill of the art after the following description has been read and understood.

FIG. 1 is an exploded side view of the fortified cannulated screw of the present invention with the lumen shown in phantom.

FIG. 2 is a side view of the fortified cannulated screw of FIG. 1 with a fortifying screw shown in place in phantom.

FIG. 3 is a side view of the fortified cannulated screw of FIG. 1 with a fortifying screw of a length different than the fortifying screw of FIG. 2 shown in place in phantom.

FIG. 4 is an end view of a fortified cannulated screw of FIG. 1 from the proximal end.

FIG. 5 is an end view of a fortified cannulated screw of FIG. 1 from the distal end.

FIG. 6 is a side view of the fortifying screw of the fortified cannulated screw of FIG. 1.

FIG. 7 is a side view of an alternate embodiment of the fortified cannulated screw of the present invention.

### DETAILED DESCRIPTION OF THE INVENTION

In order that the invention may be clearly understood and readily carried into effect, preferred embodiments of the invention will now be described, by way of example only and not to limit the invention, with reference to the accompanying drawings. The fortified cannulated screw of the present invention is shown in the drawings generally labeled 10.

The fortified cannulated screw 10 has a proximal end 12, a distal end 14, a lumen 16 and a shaft 18 having an outer surface 20. The shaft 18 extends from the proximal end 12 to the distal end 14 and contains the lumen 16.

The proximal end 12 of the fortified cannulated screw 10 preferably includes a head 22. The head 22 typically has a slightly larger diameter than the diameter of the shaft 18. In a preferred embodiment of the head 22, the head 22 includes means for interacting with a tool for screwing the fortified cannulated screw 10 into the bone. An example of such means is screw slot 24 formed in the lumen 16 at the proximal end 12. The screw slot 24 allows a screw driving tool (not shown) to be mated with the screw slot 24 so that as the screw driving tool rotates, the fortified cannulated screw 10 will also rotate.

In a preferred embodiment of the fortified cannulated screw 10, the screw slot 24 has the form of a hexagonally shaped slot and the screw driving tool is a hexagonal shaped device designed to conformally mate with the screw slot 24. Although a hexagonally shaped slot and correspondingly shaped screw driving tool has been described as a way to cause the fortified cannulated screw 10 to rotate, other means of the head 22 interacting with a rotatable tool, including but not limited to shapes of slots other than hexagonal and slots to receive flat bladed or cross point screw driver heads and forming the head 22 with a bolt head configuration and shape to interact with a socket, may also be used as will be clear to those skilled in the art.

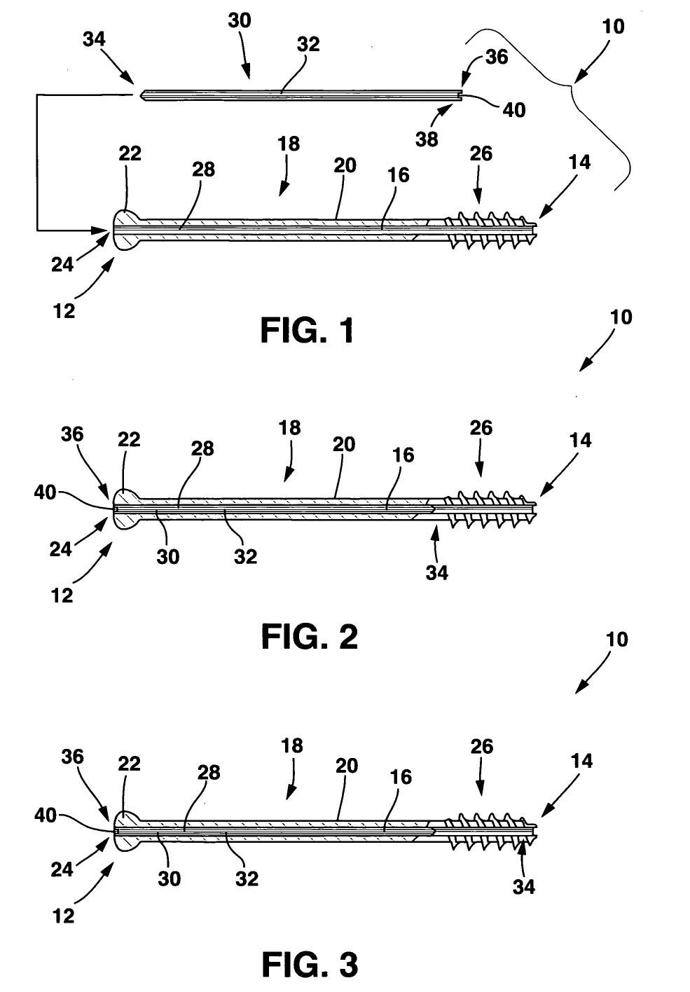

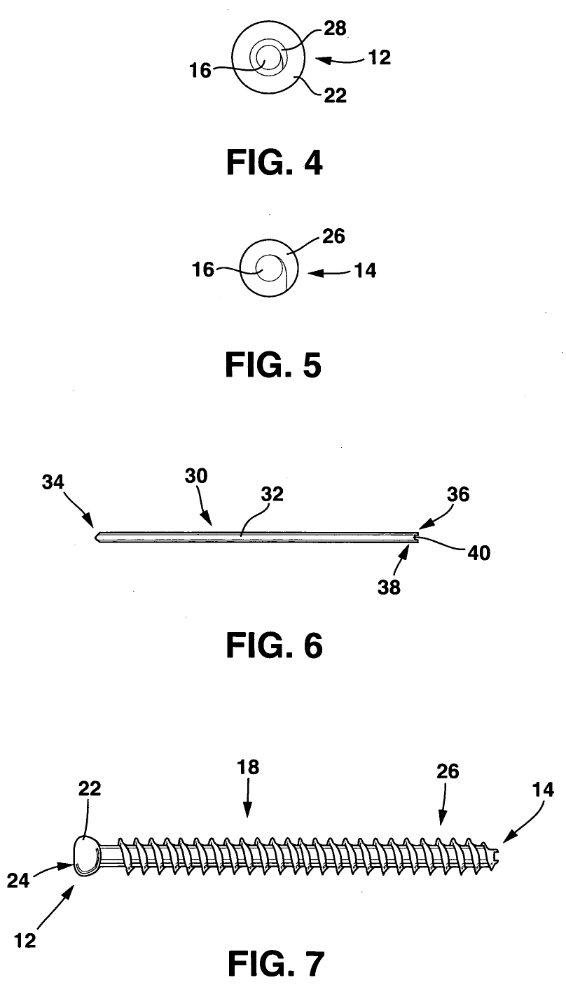

An outer screw thread 26 extends from the distal end 14 toward the proximal end 12 along the outer surface 20 and may extend partially (FIGS. 1-3) or entirely (FIG. 7) from the distal end 14 to the proximal end 12. The outer screw thread 26 is preferably of the self-reaming and self-tapping variety. This allows the fortified cannulated screw 10 to be directly placed over a guide wire or guide pin without separate reaming and tapping operations. However, outer screw thread 26 may also be of any of the well-known screw thread types as is well understood in the art. Where the outer screw thread 26 is not of the self-reaming or self-tapping variety, a borehole would first be created in the bone using conventional reaming or tapping techniques. Thereafter the fortified cannulated screw 10 is threaded into place in the borehole. It is most preferable but not essential that the outer screw thread 26 be of a type that allows for reverse cutting in order to allow the fortified cannulated screw 10 to be removed and also to prevent micro fracturing of the bone around the outer screw thread 26 as is well understood in the art.

The lumen 16 extends from the distal end 14 to the proximal end 12 and has a diameter capable of receiving a guide wire or guide pin into the lumen 16 from the distal end 14 toward the proximal end 12. For example, a typical guide wire has a diameter of about 2.0 mm and a typical guide pin has a diameter of about 3.2 mm. Consequently the diameter of the lumen 16 should be slightly larger than the diameter of the guide wire or guide pin with which the fortified cannulated screw 10 is to be used.

The lumen 16 also includes a lumen thread 28 that extends along at least a portion of the length of the lumen 16 around the inner surface of the lumen 16. In a preferred embodiment of the fortified cannulated screw 10, the lumen thread 28 extends entirely from the proximal end 12 to the distal end 14. However other embodiments of the fortified cannulated screw 10 may have a lumen thread 28 extending only a partial distance from the proximal end 12 towards the distal end 14. Further, the lumen thread 28 may be displaced from the ultimate proximal end 12 toward the distal end 14. The lumen thread 28 accommodates and mates with a fortifying screw 30 having a fortifying screw thread 32. As a result, the lumen thread 28 and the fortifying screw thread 32 must match and allow the fortifying screw 30 to be screwed into the lumen 16 through the interaction of the fortifying screw thread 32 and the lumen thread 28.

In the embodiment of the fortified cannulated screw 10 where a guide wire extends entirely through the lumen 16, the open diameter of the lumen thread 28 should be large enough to allow the guide wire to pass through the lumen thread 28. In the embodiment of the fortified cannulated screw 10 where a guide pin extends into the lumen 16 from the distal end 14, if the guide pin extends into the part of the lumen 16 containing the lumen thread 28, the open diameter of the lumen thread 28 should also be large enough to accommodate the guide pin. Where the guide pin does not extend into the part of the lumen 16 containing the lumen thread 28, the open diameter of the lumen thread 28 need not be large enough to accommodate the guide pin.

The fortifying screw 30 includes a distal end 34 and a proximal end 36. The fortifying screw 30 typically will have a screw head 38 located at the proximal end 36 to allow the fortifying screw 30 to be screwed into the lumen thread 28 as will be described hereafter. The screw head 38 may have a slightly larger diameter than the diameter of the fortifying screw thread 32 or may have the same diameter as the fortifying screw thread 32 and simply be the most proximal end 36 of the fortifying screw 30.

The fortifying screw 30 includes means for interacting with a tool for screwing the fortifying screw 30 into the lumen 16. In a preferred embodiment of the screw 38, the screw head 38 includes this means for interacting with a tool for screwing the fortifying screw 30 into the lumen 16. An example of such means is a fortifying screw head slot 40 formed in the screw head 38. The fortifying screw head slot 40 allows a flat screwdriver blade (not shown) to be mated with the fortifying screw head slot 40 so that as the flat screwdriver blade rotates, the fortifying screw 30 will also rotate. Although a fortifying screw head slot 40 has been described as a way to cause the fortifying screw 30 to rotate, other means of the screw head 38 interacting with a rotatable tool may be used as will be clear to those skilled in the art. Where there is no screw head 38, the means for interacting with a tool for screwing the fortifying screw 30 into the lumen 16 is located in the proximal end 34 of the fortifying screw 30.

The fortified cannulated screw 10 and fortifying screw 30 are preferably made of strong, resilient and durable biocompatible materials such as medical grade stainless steel, nitenol or titanium. Although these materials are metals, non-metallic materials such as ceramic or composite materials may also be used as will be clear to those skilled in the art.

The following dimensions are given as examples of the fortified cannulated screw 10 and the fortifying screw 30 and are not intended to limit the size of the fortified cannulated screw 10 and the fortifying screw 30 or the size or relative sizes of the components of each. The length of the fortified cannulated screw 10 would typically be from about 30 mm to about 150 mm. The diameter of the shaft 18 would typically be between about 3 and 8 mm. Typical lengths of the outer screw thread 26 are between about 16 mm and 32 mm. Where the shaft 18 has an outer diameter of about 4.0 mm, the head 22 might have a diameter of about 8.0 mm and the outer screw thread 26 may have an outer diameter of about 7.0 mm. For a guide wire having a typical 2 mm diameter, the lumen 16 would have a diameter about slightly larger than 2 mm. A typical fortifying screw 30 would have a length of between about 30 mm to about 150 mm and have an outside diameter to match the lumen 16 of about 2 mm.

As mentioned, these dimensions have been given as examples of typical cannulated fortified cannulated screws 10 and its accompanying fortified screw 24. However, the dimensions may be changed or modified as will be clear to those skilled in the art. For example, in a smaller fortified cannulated screw 10, the dimensions cited above might all be diminished proportionately. Conversely, in a larger fortified cannulated screw 10, any or all of the dimensions given above may be increased, again as the will be clear to those skilled in the art.

The fortified cannulated screw 10 of the present invention is believed to be useful where any cannulated screw would be used including but not limited to fractures of the intracapsular hip, femoral condyles, tibial condyles, ankle, acetabulum, pelvis, in the treatment of sipped capital femoral epiphysis and in fusing bones of the ankle or vertebrae. It may also be used in other areas where accurate placement of a strong cancellous screw is required.

This new fortified cannulated screw 10, with the fortifying screw 30, combines the advantages of traditional cannulated screw in its ability to be accurately placed using a guide wire or a guide pin with the strength of traditional solid screw.

In use wherever used, the fortified cannulated screw 10 is placed into a desired location in bone. This is preferably done using a guide pin or guide wire to accurately locate the fortified cannulated screw 10. When a guide pin or guide wired is used, the guide pin or guide wire is first placed in the bone using conventional techniques. The guide pin or guide wire is then placed in the distal end 14 of the lumen 16 and the fortified cannulated screw 10 is moved along the guide wire or guide pin so that the distal end 14 of the fortified cannulated screw 10 comes in contact with bone.

Where the fortified cannulated screw 10 has a self-reaming or self-tapping outer screw thread 26, the fortified cannulated screw 10 is screwed into the bone by the interaction between the screw driving tool and the screw slot 24 of the lumen 16 so that the outer screw thread 26 cuts its own screw thread channel into the bone. Where the fortified cannulated screw 10 does not have a self-reaming or self-tapping outer screw thread 26, a borehole is first be cut in the bone using traditional reaming or tapping devices including such devices that move along guide wires or guide pins. Thereafter the fortified cannulated screw 10 is moved along the guide wire or guide pin so that the distal end 14 of the fortified cannulated screw 10 moves into contact with the borehole. Once the fortified cannulated screw is in contact with the borehole, the fortified cannulated screw 10 is screwed into the bone where the outer screw thread 26 cuts a screw thread channel into the bone around the borehole.

When the fortified cannulated screw 10 is in the desired position in the bone, the fortifying screw 30 is screwed into the lumen threads 24 until the proximal end 36 of the fortifying screw 30 comes into seated engagement with the proximal end 12 of the fortified cannulated screw 10. As a result, the fortified cannulated screw 10 with the fortifying screw 30 in place has a strength about equal to the strength of a solid screw of the same dimensions.

The fortified cannulated screw 10 is then tightened in position to compress the bone fragments and hold the reduction in place. Because the fortified kingly screw 10 as the fortifying screw 30, the overall fortified cannulated screw 10 is strong enough to allow significant weight-bearing to be placed on the fortified cannulated screw 10 so that the patient may be mobile thereby aiding in the healing bone healing process.

While the above description contains many specificities, these should not be construed as limitations on the scope of the invention, but rather as examples of preferred embodiments thereof. As a result, the description contained herein is intended to be illustrative and not exhaustive. Many variations and alternatives of the described technique and method will occur to one of ordinary skill in this art. Variations in form to the component pieces described and shown in the drawings may be made as will occur to those skilled in the art. Further, although certain embodiments of an fortified cannulated screw 10 have been described, it is also within the scope of the invention to add other additional components such as surface coatings or to modify the components, particularly to adapt the fortified cannulated screw 10 for use with specific bones. Also, many variations in the shape or relative dimensions of the components will occur to those skilled in the art and still be within the scope of the invention.

All these alternatives and variations are intended to be included within the scope of the attached claims. Those familiar with the art may recognize other equivalents to the specific embodiments described herein which equivalents are also intended to be encompassed by the claims attached hereto. As a result, while the above description contains may specificities, these should not be construed as limitations on the scope of the invention but rather as examples of different embodiments thereof.

What I claim is:

1. A fortified cannulated screw comprising:
a shaft having a proximal end, a distal end, a lumen, the shaft having an outer surface wherein the lumen extends from the distal end to the proximal end and the lumen has a lumen thread that extends along at least a portion of the length of the lumen around an inner surface of the lumen;
an outer screw thread extending from the distal end toward the proximal end along the outer surface;
a fortifying screw having a distal end, a proximal end, a fortifying screw thread and means for interacting with a tool for screwing the fortifying screw into the lumen wherein the lumen thread and the fortifying screw thread match to allow the fortifying screw to be screwed into the lumen through the interaction of the fortifying screw thread and the lumen thread.

2. The fortified cannulated screw of claim 1 wherein the proximal end of the fortified cannulated screw includes a head.

3. The fortified cannulated screw of claim 2 wherein the head has a slightly larger diameter than the diameter of the shaft.

4. The fortified cannulated screw of claim 2 wherein the head includes means for interacting with a tool for screwing the fortified cannulated screw into a bone.

5. The fortified cannulated screw of claim 4 wherein the means for interacting is screw slot formed in the lumen at the proximal end.

6. The fortified cannulated screw of claim 5 wherein the screw slot has the form of a hexagonally shaped slot and the screw driving tool is a hexagonal shaped device designed to conformally mate with the screw slot.

7. The fortified cannulated screw of claim 5 wherein the screw slot is chosen from the group consisting of slots to receive flat bladed or cross point screw driver heads and forming the head with a bolt head configuration and shape to interact with a socket.

8. The fortified cannulated screw of claim 1 wherein the outer screw thread extends partially from the distal end toward the proximal end along the outer surface.

9. The fortified cannulated screw of claim 1 wherein the outer screw thread extends substantially entirely from the distal end toward the proximal end along the outer surface.

10. The fortified cannulated screw of claim 1 wherein the outer screw thread is of the self-reaming and self-tapping variety.

11. The fortified cannulated screw of claim 1 wherein the lumen has a diameter capable of receiving a guide wire or guide pin into the lumen from the distal end toward the proximal end.

12. The fortified cannulated screw of claim 1 wherein the lumen thread extends entirely from the proximal end to the distal end.

13. The fortified cannulated screw of claim 1 wherein the lumen thread extends only a partial distance from the proximal end towards the distal end.

14. The fortified cannulated screw of claim 1 wherein the lumen thread is displaced from the ultimate proximal end toward the distal end.

15. The fortified cannulated screw of claim 1 wherein the open diameter of the lumen thread is large enough to allow a guide wire to pass through the lumen thread.

16. The fortified cannulated screw of claim 1 wherein the open diameter of the lumen thread is large enough to allow a guide pin to pass through the lumen thread.

17. The fortified cannulated screw of claim 1 wherein the fortifying screw has a screw head located at the proximal end of the fortifying screw to allow the fortifying screw to be screwed into the lumen thread.

18. The fortified cannulated screw of claim 17 wherein the screw head has a slightly larger diameter than the fortifying screw thread.

19. The fortified cannulated screw of claim 17 wherein the screw head has the same diameter as the fortifying screw thread.

20. The fortified cannulated screw of claim 17 wherein the means for interacting with a tool for screwing the fortifying screw into the lumen is located in the screw head.

21. The fortified cannulated screw of claim 1 wherein the means for interacting with a tool for screwing the fortifying screw into the lumen is fortifying screw head slot that allows a flat screwdriver blade to be mated with the fortifying screw head slot so that as the flat screwdriver blade rotates, the fortifying screw will also rotate.

22. A fortified cannulated screw comprising:
a shaft having a proximal end, a distal end, a lumen, the shaft having an outer surface wherein the lumen extends from the distal end to the proximal end, has a lumen thread that extends along at least a portion of the length of the lumen around an inner surface of the lumen and has a diameter capable of receiving a guide wire or guide pin into the lumen from the distal end toward the proximal end;
an outer screw thread extending from the distal end toward the proximal end along the outer surface wherein the outer screw thread is of the self-reaming and self-tapping variety;
a fortifying screw having a distal end, a proximal end, a fortifying screw thread and means for interacting with a tool for screwing the fortifying screw into the lumen wherein the lumen thread and the fortifying screw thread match to allow the fortifying screw to be screwed into the lumen through the interaction of the fortifying screw thread and the lumen thread.

23. A method of fortifying a bone screw comprising the steps of:
(a) providing a fortified cannulated screw comprising:
(i) a shaft having a proximal end, a distal end, a lumen, the shaft having an outer surface wherein the lumen extends from the distal end to the proximal end and the lumen has a lumen thread that extends along at least a portion of the length of the lumen around an inner surface of the lumen;
(ii) an outer screw thread extending from the distal end toward the proximal end along the outer surface;
(iii) a fortifying screw having a distal end, a proximal end, a fortifying screw thread and means for interacting with a tool for screwing the fortifying screw into the lumen wherein the lumen thread and the fortifying screw thread match to allow the fortifying screw to be screwed into the lumen through the interaction of the fortifying screw thread and the lumen thread;
(b) placing the distal end of the fortifying screw in the lumen at the proximal end of the shaft; and
(c) rotating the fortifying screw so that the fortifying screw threads engage with the lumen threads and move the fortifying screw into the lumen until the proximal end of the fortifying screw is proximate the proximal end of the shaft.
